(19) United States
(12) Patent Application Publication
(10) Pub. No.: US 2008/0188676 A1
(43) Pub. Date: Aug. 7, 2008

(54) METHODS OF ROBUST AND EFFICIENT CONVERSION OF CELLULAR LIPIDS TO BIOFUELS

(76) Inventors: Gregory A. Anderson, Takaka (NZ); Vincent V. Cunetto, Hillsboro, OR (US)

Correspondence Address: WILSON SONSINI GOODRICH & ROSATI, 650 PAGE MILL ROAD, PALO ALTO, CA, 94304-1050, US

(21) Appl. No.: 11/855,992

(22) Filed: Sep. 14, 2007

Related U.S. Application Data
(60) Provisional application No. 60/844,907, filed on Sep. 14, 2006.

(51) Int. Cl. C11B 1/00; C12M 1/00

(52) U.S. Cl. 554/21; 554/8; 435/289.1

(57) ABSTRACT

Methods, vessels, and systems are provided for processing lipids contained in biomass, such as organisms grown in aqueous media or wastes in aqueous media, to produce fatty acid esters as components of a fuel, such as biofuels. The methods described herein are able to efficiently convert cellular lipids to biofuels from lipid-containing biomass such as algae.

### CROSS-REFERENCE

This application claims the benefit of U.S. Provisional Application No. 60/844,907, filed Sep. 14, 2006, which application is incorporated herein by reference.

### BACKGROUND OF THE INVENTION

In recent years there has been a considerable research effort directed towards finding alternatives to petroleum based fuels that utilize biologically derived starting materials. Along with work to develop gasoline substitutes, such as ethanol, butanol, and pyrolytically-formed, biomass-derived hydrocarbons, there has been a similarly active pursuit of diesel fuel replacements.

Alkyl esters of fatty acids, 8 to 24 carbon atoms in length, have been widely proposed as desirable replacements for petroleum-based diesel engine fuels. These blends of fatty acid esters, collectively commonly known as biodiesel are typically produced via transesterification reactions, involving nature-derived lipids and short chain alcohols as reactants.

There are several methods currently used for the production of these fuel esters, the details of which have been extensively published. Briefly, the most common techniques involve alkaline, acid, or mineral catalysis, of either homogeneous or heterogeneous nature. The reactions are typically quite sensitive to the degree of lipid purity, such that lipid containing feedstocks possessing excessive content of water, free fatty acids, or cellular debris are considered to be unsuitable as starting materials, due to reduced reaction rate, catalyst inactivation, or poor product yield.

One potential lipid source with promise as a biodiesel feedstock is the group consisting of microalgae, macroalgae, fungi, and bacteria. These organisms grow rapidly, are readily cultured in aqueous media, and can attain high ratios of biomass lipid production for a given volume. They can yield over 50% of cell weight as lipid-like constituents. An additional advantage lies in the possibility of growing these species on land deemed otherwise unsuitable for oil seed or food production.

This approach to lipid and subsequent fuel production has proven to be challenging in practice, however, particularly in regards to actual lipid isolation and conversion of cell-bound lipids to fuels.

Typically, cellular material is concentrated from dilute culture media by means of filtration, flocculation, or centrifugation. In most cases, the isolated biomass needs to be dried to effect successful lipid extraction. The extraction is normally accomplished with solvents. After solvent removal, the isolated oil or fat can be subjected to transesterification in order to produce alkyl esters suitable as diesel engine fuels.

It would be advantageous, in the interest of conserving energy, to obviate the need for excessive concentration, drying, or solvent extraction of the cellular material during the fuel production sequence.

### SUMMARY OF THE INVENTION

In an aspect of the invention, a method of producing fatty acid esters comprises reacting a composition comprising cellular material in a reactor. The temperature and pressure within the reactor are elevated such that the cellular material is destructible and the components of the cellular material form an aqueous phase and an oily phase. In many embodiments, the reaction conditions according to the temperature and pressure conditions are near critical or supercritical reaction conditions. An alcohol is then reacted with the oily phase from the first reaction, thereby producing fatty acid esters.

In an another aspect of the invention, a method of producing fatty acid esters comprises reacting a composition comprising cellular material in a reactor. The temperature and pressure within the reactor are elevated that the cellular material is destructible and the components of the cellular material form an aqueous phase and an oily phase. The aqueous phase can then be separated from the oily phase, and the oily phase can then reacted with an alcohol, thereby producing fatty acid esters.

The separation of the aqueous phase from the oily phase can be conducted by a variety of methods, including those well known in the art. Examples of separation methods include, but are not limited to, settling, gravity separation, centrifugal separation, filtration, and extraction.

In an aspect of the invention, a method of producing fatty acid esters comprises reacting a composition comprising cellular material in a reactor in the presence of an aqueous solution. The temperature and pressure within the reactor are elevated such that the cellular material is destructible and the components of the cellular material form an aqueous phase and an oily phase. An alcohol is then reacted with the oily phase from the first reaction, thereby producing fatty acid esters.

The aqueous solution can be water that can be reacted with the composition comprising cellular material. The aqueous solution can be between 5% and 90% of the total reaction composition when reacting the aqueous solution with the composition comprising cellular material.

In an aspect of the invention, a method of producing fatty acid esters comprises reacting a composition comprising cellular material in the presence of alcohol in a reactor. The temperature and pressure within the reactor are elevated such that the cellular material can be destructed. If destruction of the cellular material occurs, the cellular material can react under the conditions of elevated temperature and pressure to form an aqueous phase and an oily phase. Concurrently with, or after, the cellular material forms an aqueous and an oily phase, the alcohol can react with the oily components of the cellular material, thereby producing a fatty acid ester.

In another aspect, a method is disclosed comprising a one step method as described herein, wherein the reactor contains a porous structure. The porous structure can be reticulated foam.

The cellular material may be any lipid-containing biomass such as biomass from animals, plants, fungi, and microorganisms, such as microalgae, macroalgae, bacteria, diatoms, and protozoa. Examples of cellular materials from animals include, but are not limited to, fat-containing tissues from animals such as chickens, lambs, sheep, cows, rat, mice, whales, and fish. Examples of cellular materials from plants include, but are not limited to, biomass from plants such as trees, grass, agricultural crops, grains crop residues, and grains. In some embodiments, the cellular material comprises intact cells. In other embodiments, cellular material has been dried. Optionally, the cellular material comprises at least 5%, 10%, 30%, 50%, 70% intact cells w/w based the concentration of the cellular material.

In some embodiments, the composition comprising the composition may contain at least 1%, 5%, 10%, 20%, or 50% water by weight. In some embodiments, the composition comprising the cellular material may contain between 1-50%, 5-40%, 10-90% water w/w based on the total weight of the composition. In some embodiments, the composition contains between 10% and 90% cellular material w/w based on the total weight of the composition.

The elevated temperature and pressure within a reactor for destructing cellular material of the composition may approach or may be at supercritical conditions. For example, the temperature may be elevated to between 180° C. and 450° C., and the pressure can be elevated to between 0.5 MPa and 40 MPa. In an embodiment, the temperature is elevated to between about 320° C. and 370° C. In a further embodiment, the temperature is elevated to 350° C. In an embodiment, the pressure is elevated to 20 MPa.

In an embodiment of a method, the oily phase comprises at least one of fatty acids, monoglycerides, diglycerides, and triglycerides. The oily phase may be reacted with the alcohol at a near critical or supercritical reaction condition.

The alcohol reacted with the oily phase can have 1 to 20 carbon atoms. In some embodiments, the alcohol is methanol or ethanol.

The fatty acid ester produced by a method of the invention may be a fatty acid methyl ester.

In an embodiment, any reaction of the invention is carried out in the presence of a co-solvent. Examples of co-solvents include, but are not limited to, carbon dioxide, nitrous oxide, sulfur dioxide, sulfur hexafluoride, alkanes and alkenes containing between 1 and 20 carbon atoms, alkyl halides, aromatic hydrocarbons, silicones, ethers, amines, alkyl oxides, and esters.

Reactions of embodiments of methods of the invention can be carried out in the presence of a catalyst. Examples of catalysts include, but are not limited to, inorganic or organic acids or bases, metals or their oxides, silicates, carbonates or other salts of elements such as the alkali elements including aluminum, magnesium, calcium, titanium, hafnium, nickel, silicon and zirconium.

In an embodiment, fatty acid esters produced by a method of the invention can be purified for their use in various products, such as biodiesel.

A vessel is provided herein in which any of the methods of the invention can be carried out. In many embodiments, the vessel is capable of withstanding elevated temperatures and pressures. The vessel can be of multiple geometries, such that the reaction can occur in the fashion known as “batch” processing, or as “continuous flow” processing.

The invention also provides a system comprising a reactor containing a composition comprising cellular material, a means for elevating the temperature and pressure within the reactor, and an outlet for collecting fatty acid esters.

The reactor can be any reactor, vessel, or device capable of carrying out at least one portion of any method the invention described herein. The reactor can also be a vessel of the invention.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the invention will be obtained by reference to the following detailed description that sets forth illustrative embodiments, in which the principles of the invention are utilized, and the accompanying drawings of which:

FIG. 1 illustrates a method of conversion of lipid containing cellular material to fatty acid esters under conditions of elevated temperature and pressure comprising a separation step.

FIG. 2 demonstrates the conversion of lipid containing cellular material to fatty acid esters in an individual vessel in the presence of alcohol.

FIG. 3 illustrates a method of converting lipid containing cellular material into a fuel.

### DETAILED DESCRIPTION OF THE INVENTION

While preferred embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

This invention pertains to a method for transforming cellular biomass, such as algae, diatoms, protozoa, bacteria, fungi, and waste of cellular origin, into useful products. The invention also can include a method for transforming biomass into fuel additives or fuel products, such as biodiesel.

The materials used in the methods may also include waste products, such as leaves and grass clippings, rice hulls, bagasse, seaweed, milling waste, cotton waste, and animal waste. Disposal of these wastes is currently expensive, and can create environmental problems.

The supercritical reaction conditions referred to herein refers to the following. Fluids in the supercritical condition show a behavior different from the normal states of liquid or gas. A fluid in the supercritical condition is a non-liquid solvent having a density approximate to that of liquid, a viscosity approximate to that of gas, and a thermal conductivity and a diffusion coefficient which are intervenient between those of gas and of liquid. The low viscosity and high diffusion of supercritical fluids favor mass transfer therein, and its high thermal conductivity enables high thermal transmission. Because of such a special condition, the reactivity in the supercritical condition is higher than that in the normal gaseous or liquid state and thus esterification and/or transesterification is promoted. One of the most important properties of supercritical fluids is their solvating properties are a complex function of their pressure and temperature, independent of their density.

The near critical condition referred to herein refers to conditions with proximity to the supercritical conditions.

The invention contemplates a method of generating a fuel, such as biodiesel, from fatty acid esters produced in one or more embodiments of the invention disclosed herein. Examples of fatty acid esters for use in biofuel for diesel engine include, but are not limited to, fatty acid methyl ester, fatty acid ethyl ester, fatty acid isopropyl ester, fatty acid isobutyl ester and the like.

The fuel production methods and vessels described herein provide an economical and environmentally-friendly means of handling of organisms grown in aqueous media or wastes in aqueous media. This renewable energy source can be used as a process load. Energy can be generated in quantities sufficient to meet the steam load of a processing plant after start-up, without the need for any added auxiliary fuel. The energy produced can additionally or alternately be commercially sold and/or used to generate electricity. Alternatively, some or all of the biofuel, can be sold, thus providing operational flexibility.

The systems and methods described herein not only provides a profitable means to process lipids contained in biomass, such as organisms grown in aqueous media or wastes in aqueous media, but also allows the resulting commodity, i.e., energy, to be used as an alternative power source to help reduce dependence on fossil fuels. Reducing dependence on fossil fuels, particularly on foreign oil supplies, is of particular importance in the present turbulent political and economic climate. Additionally, with energy demands expected to increase significantly in the future, use of renewable energy sources will become increasingly important.

The methods describe herein can form alkyl esters via a two or one step method which can utilize a composition comprising a cellular material, or a composition comprising an aqueous solution containing a cellular material or oily/fatty slurries of the cellular material.

The methods of the invention can utilize water and alcohol or alcohol alone in a state of enhanced energy to perform cell destruction, hydrolysis, and concurrent or subsequent alkyl ester formation. The methods can also comprise co-solvents and/or catalysts.

When aqueous slurries of lipid containing cellular material, such as algae, are subjected to elevated temperature and pressure, such as near critical or supercritical conditions, hydrolysis of the lipid-like cellular components occurs rapidly, and the resulting free fatty acids or oily phase can be distinct from the aqueous portion or aqueous phase of the reaction mixture.

The resulting oily phase can be separated from the aqueous portion and then subjected to transesterification in the same reactor or vessel or in a different reactor. A separation may or may not be executed between the two reactions. Alcohol can be added to the reactor for transesterification after the hydrolysis of the lipid-like cellular components.

In an aspect of the invention, a method of producing fatty acid esters comprises reacting a composition comprising cellular material in a reactor. The temperature and pressure within the reactor are elevated such that the cellular material is destructible and the components of the cellular material form an aqueous phase and an oily phase. In many embodiments, the reaction conditions according to the elevated temperature and pressure conditions are near critical or supercritical reaction conditions. An alcohol is then reacted with the oily phase from the first reaction, thereby producing fatty acid esters.

In an embodiment, a method of the invention is disclosed herein that eliminates the need to isolate lipid components from cellular materials in order to produce fatty acids and their alkyl esters. Under certain conditions of enhanced thermal activity, cell bound lipids react rapidly and completely to form either free fatty acids or alkyl esters thereof. Additionally, the free fatty acids can be subsequently esterified by a number of well-known methods to form alkyl esters, suitable as diesel engine fuel.

The cellular material may be any lipid-containing biomass such as biomass from animals, plants, fungi, and microorganisms, such as microalgae, macroalgae, bacteria, diatoms, and protozoa. Examples of cellular materials from animals include, but are not limited to, fat-containing tissues from animals such as chickens, lambs, sheep, cows, rat, mice, whales, and fish. Examples of cellular materials from plants include, but are not limited to, biomass from plants such as trees, grass, agricultural crops, grains crop residues, and grains. In some embodiments, the cellular material comprises intact cells.

In some embodiments, the intact cells are grown in an aqueous medium. In other embodiments, cellular material has been dried. The composition comprising cellular material can contain at least 1%, 5%, 10%, 20%, or 50% water by weight. In an embodiment, the composition comprising cellular material can contain between 10% and 70% water by weight.

In a typical application, an aqueous slurry of cells, such as a microalgal or bacterial paste, is subjected to substantially increased temperature and pressure, with or without catalytic activators or external energy supplementation (ultrasonic, microwave, etc.) in order to disintegrate the structural components of said cell, and hydrolyse carbohydrates, oily esters, and proteins. This is a mild reaction which results in high yields of desirable components.

Pressures during the reaction can range from 0.5 to 50 MPa and, in a preferable embodiment, from 6 to 25 MPa. Temperatures during the reaction can range from 80° C. to 450° C. and, in a preferable embodiment, from 250° C. to 360° C. These conditions are approaching or are within the range which is described as near critical or supercritical conditions.

In an embodiment of a method, the oily phase comprises at least one of fatty acids, monoglycerides, diglycerides, and triglycerides. The oily phase can be reacted with the alcohol at a near critical or supercritical reaction condition.

The alcohol reacted with the oily phase can have 1 to 20 carbon atoms. In some embodiments, the alcohol is methanol or ethanol.

The chosen alcohol can be mixed with the oily phase, in a molar ratio of alcohol to fatty acids ranging from 1 part alcohol to 1 part oily phase up to 80 parts alcohol to 1 part oily phase. The alcohol can be added to the reactor or vessel under conditions of pressure and temperature such as those described herein. The reaction is allowed to proceed until substantially complete. Such time can range from 1 minute to 60 minutes and, in a preferable embodiment, from 4 minutes to 18 minutes. After the reaction to produce fatty acid esters ends, the reactants can be removed from the reactor and separated from excess alcohols, co-solvents, and/or water in a manner consistent with known isolation and purification techniques to obtain the fatty acid esters.

The fatty acid ester produced by a method of the invention can be a fatty acid methyl ester. The fatty acid ester produced by the methods described herein can be used in fuels such as a fuel for diesel engine, base oil for lubricant oil, an additive for fuel oil and the like by itself or in admixture with other components according to the requirements derived from the use.

As may be well known to those skilled in the art, a multitude of techniques exist for the conversion of organic or fatty acids and oils to their esters, and that many secondary esterification methods could be employed to convert the oily phase obtained from the hydrolysis of the cellular material as described above to their alkyl esters. Such classic techniques include, but are not limited to, reactions of fatty acids with alcohols and alkenes under the influence of a diverse array of homogeneous and heterogeneous catalysts and dehydration agents. Lower temperatures and pressures can be employed by either supplementing the reaction with external energy sources such as microwave or ultrasonic energy, lowering the activation energy of the reaction via catalysis, or lowering the critical point of the solvent system through incorporation of additional solvents.

A supercritical transesterification reaction comprises either the oil or fat or fatty acid or alcohol in a supercritical condition. The mixture of these components can be in a near critical or supercritical condition. In the embodiments of the invention described herein, an additional solvent may be included with the reaction mixture within the reaction vessel and can be in a near critical or supercritical condition. An additional solvent, or co-solvent, can often lower the temperature and pressure needed to make the reaction enter the supercritical reaction conditions. Examples of the additional solvent include, but are not limited to, carbon dioxide, nitrous oxide, sulfur dioxide, sulfur hexafluoride, alkanes and alkenes containing between 1 and 20 carbon atoms, alkyl halides, aromatic hydrocarbons, silicones, ethers, amines, alkyl oxides, and esters.

In an embodiment, any reaction of the invention can be carried out in the presence of a co-solvent. In some methods of the invention, the severity of the temperature and pressure parameters of the reaction conditions can be reduced by addition of a co-solvent to the reaction vessel. Various gases and liquids that can serve as examples of co-solvents include, but are not limited to, carbon dioxide, nitrous oxide, sulfur dioxide, sulfur hexafluoride, alkanes and alkenes containing between 1 and 20 carbon atoms, alkyl halides, aromatic hydrocarbons, silicones, ethers, amines, alkyl oxides, and esters.

In the method wherein a co-solvent can be used, it can be desirable to choose a material which will allow for ready separation of the co-solvent and fatty acids from the aqueous reaction mixture, and subsequent recovery of the co-solvent.

Reactions of embodiments of methods of the invention can be carried out in the presence of a catalyst. When a nickel-containing solid catalyst is used in the invention, it can be preferable to carry out the reaction under conditions in an oil or fat and/or the alcohol and/or solvent are in a supercritical condition. Examples of catalysts include, but are not limited to, inorganic or organic acids or bases, metals or their oxides, silicates, carbonates or other salts of elements such as the alkali elements including aluminum, magnesium calcium, titanium, hafnium, nickel, silicon and zirconium.

In an embodiment, fatty acid esters produced by a method of the invention can be purified for their use in various products, such as biodiesel. Examples of purification methods include, but are not limited to, crystallization, distillation, chromatography, partitioning, and adsorptive processes.

In an another aspect of the invention, a method of producing fatty acid esters comprises reacting a composition comprising intact cells in a reactor. The temperature and pressure within the reactor are elevated such that the cellular material is destructible and the components of the cellular material form an aqueous phase and an oily phase. The aqueous phase can then be separated from the oily phase, and the oily phase can then reacted with an alcohol, thereby producing fatty acid esters.

The separation of the aqueous phase from the oily phase can be conducted by a variety of methods, including those well known in the art. The aqueous phase can comprise contains simple carbohydrates, amino acids, proteins, and other cellular breakdown products. The oily phase can comprise such compounds as fatty acids and monoglycerides, diglycerides, and triglycerides. Examples of separation methods include, but are not limited to, settling, gravity separation, centrifugal separation, filtration, membrane separation, and extraction. Extraction can be carried out by means of a solvent, such as hexane, dichloromethane, and ethyl acetate. As is known in the art, supercritical extraction can also be used to separated an aqueous phase from an oily phase.

In an embodiment of a method of the invention, the transesterification reaction can be in tandem with a hydrolysis reaction, by removal of the aqueous reaction product from the first step, introduction of the desired alcohol to the system, and continuation of conditions of elevated pressure and temperature within the same containment vessel.

It is inherent herein that the embodiments of methods of the invention are translatable and applicable to all the methods of the invention. For example, the cellular material of a method comprising a separation method can comprise intact cells, such as intact cells of algae, in the same manner as a method that may not comprise a separation method.

In an aspect of the invention a method of producing fatty acid esters comprises reacting a composition comprising cellular material in a reactor in the presence of an aqueous solution. The temperature and pressure within the reactor are elevated that the cellular material is destructible and the components of the cellular material form an aqueous phase and an oily phase. An alcohol is then reacted with the oily phase from the first reaction, thereby producing fatty acid esters.

In an embodiment, the aqueous solution is water that can be reacted with the composition comprising cellular material. In another embodiment, the aqueous solution is between 5% and 90% of the total reaction composition when reacting the aqueous solution with the composition comprising cellular material.

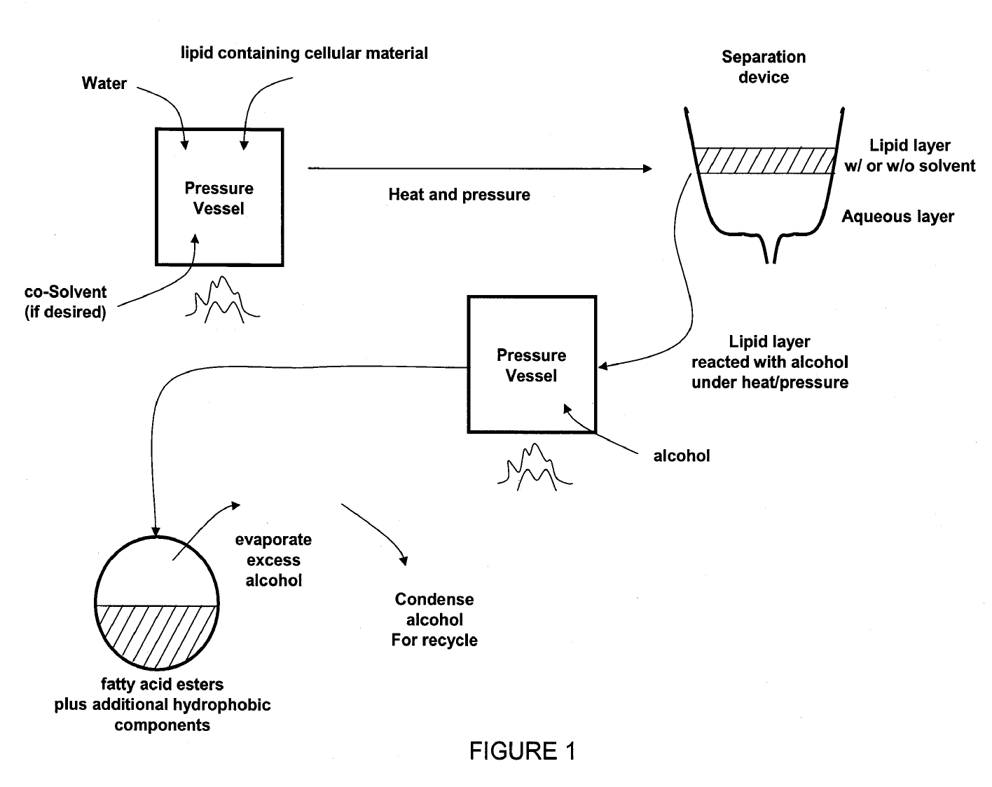

An example of some of the embodiments of the invention is illustrated in FIG. 1. The pressure vessel in FIG. 1 represents a reactor that is capable of withstanding elevated temperature and pressure. Lipid containing cellular material is deposited within the pressure vessel or reactor before or after the temperature and pressure are elevated. Water or an aqueous solution can also be deposited in the reactor as shown in FIG. 1. In addition, a co-solvent, such as carbon dioxide, can be deposited in the reactor to lower the energy requirements of a reaction. The cellular material within the pressure vessel reacts under heat and pressure for a certain period of time, as determined by a user of the method. After the time, the cellular material can form an aqueous phase and an oily phase, in this example, the aqueous phase is represented by the aqueous layer in the separation device. The oily phase, as represented by the lipid layer, naturally separates on top of the aqueous phase due to a difference in density. The aqueous layer can then be separated from the lipid layer. The separation step of the example need not be carried out before conducting the second step of the two step reaction as described herein. In the example in FIG. 1, a supercritical transesterification reaction is carried out with the lipid layer reacting with an alcohol as both are added to either the original pressure vessel or a different pressure vessel. After the supercritical transesterification reaction, the alcohol can be evaporated away in this example. The alcohol can be recycled for future method reactions. In FIG. 1, after the alcohol is evaporated, the remaining product from the starting cellular material is fatty acid esters and some other lipid components. These fatty acid esters, as demonstrated in FIG. 1, can be as fuel components.

Reacting organic compounds with near critical or supercritical aqueous solution can dramatically transform the organic compounds over short time periods (on the order of minutes to hours). The reductive process can be conducted in anaerobic or near-anaerobic conditions. The reductive process is conducted in anaerobic or near-anaerobic conditions, essentially free of any strong oxidants. Optionally, strong reducing agents or other co-reactants may be added to tailor product distributions. The method can work with a wide range of organic compounds and biomass sources, including cellulose, chitin, starches, lipids, proteins, lignin, and intact cells. The reaction of cellular material with an aqueous solution can create an aqueous phase and an oily phase. The oily phase can be put through a transesterification reaction to create fatty acid esters that allow the generation of a burnable fuel.

Another method which can be employed for the conversion of lipid containing cellular materials to fatty acid esters or fatty acid alkyl esters includes introduction of the cellular material and the desired alcohol to a containment vessel or reactor.

It is inherent herein that the embodiments of methods of the invention are translatable and applicable to all the methods of the invention. For example, the cellular material of a one-step method as described herein can comprise intact cells, such as algae, in the same manner as a two-step method described herein.

In an aspect of the invention, a method of producing fatty acid esters comprises reacting a composition comprising cellular material in the presence of alcohol in a reactor. The temperature and pressure within the reactor are elevated such that the cellular material can be destructed. If destruction of the cellular material occurs, the cellular material can reaction under the conditions of elevated temperature and pressure to form an aqueous phase and an oily phase. Concurrently with, or after, the cellular material form an aqueous and an oily phase, the alcohol can react with the oily components of the cellular material, thereby producing a fatty acid ester. This method is also referred to herein as a one step method.

Contrary to what may be expected, the destruction of a cell or cellular material and hydrolysis of cellular components can be performed concurrently with the formation of oily components by incorporating alcohols in the reaction step. For example, wet cellular mass can be reacted with a desired alcohol, while maintained at an enhanced energy state, due to the elevated temperature and pressure for a given period of time, to yield a mixture of fatty acid esters and aqueous hydrolyzed cellular components. The fatty acid esters can be used as fuel additives or fuel, such as biodiesel.

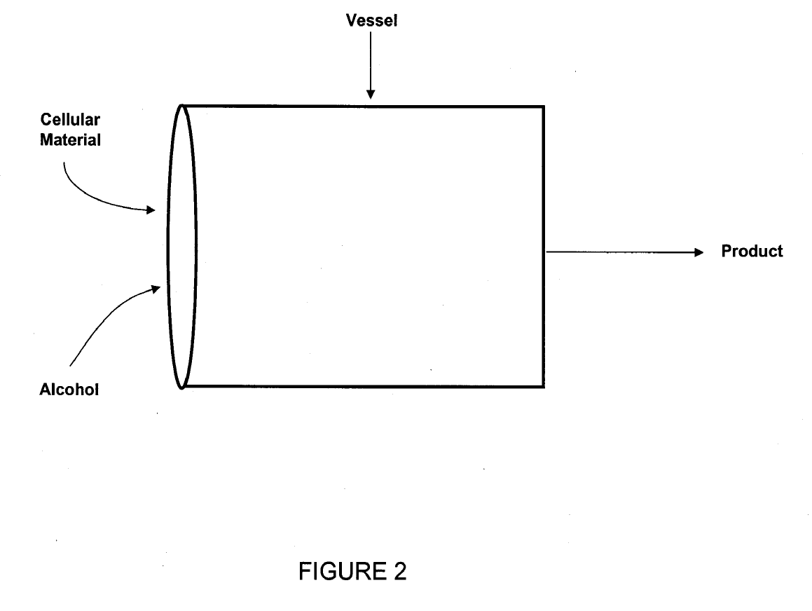

An embodiment of a one-step method of the invention is illustrated in FIG. 2. Cellular material and alcohol can be added to a vessel or reactor capable of maintaining elevated temperature and pressure reaction conditions. In some embodiments, the reaction conditions are supercritical conditions. Under the conditions of elevated temperature and pressure, the lipid components of the cellular material can react with the alcohol in a supercritical transesterification reaction. The product of such a reaction is fatty acid esters. The supercritical conditions can also destruct the cellular material into aqueous and oily phases. Hence, an aqueous material that may be useful for the production of ethanol by fermentation processes and oily components such as hydrocarbons that may be useful for fuel production can also be products of the one-step reaction method illustrated in FIG. 2.

The reaction can be conducted under the conditions described previously for cellular hydrolysis, with or without addition of a co-solvent, or co-solvents, as previously described. The reaction product can consist of a mixture of fatty acids, fatty acid alkyl esters, cellular hydrolysis and alcoholysis compounds and other cellular degradation products. Fatty acid alkyl esters predominate in the reaction product, and can be readily isolated and purified by techniques well known to those involved with chemical processes, including, but not limited to extraction, partitioning, distillation, crystallization, chromatography, and membrane treatments.

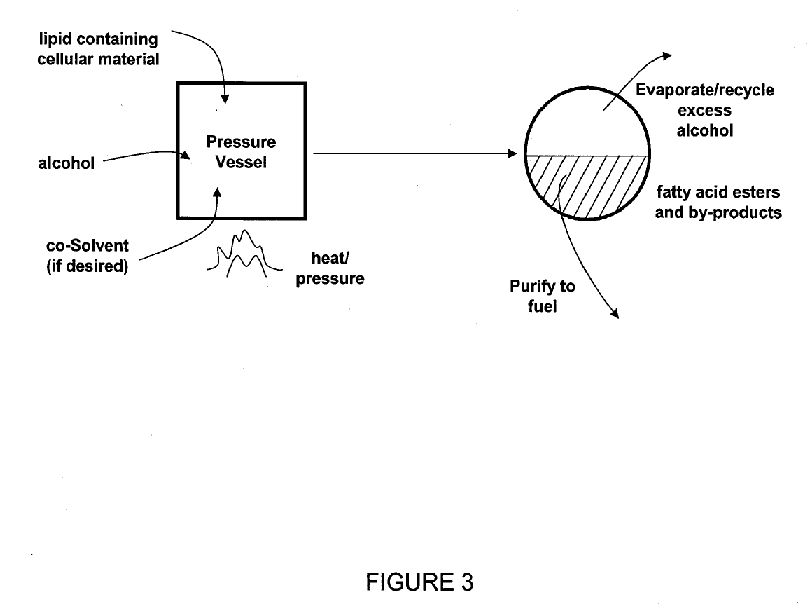

An embodiment of a one-step reaction method is demonstrated in FIG. 3. Lipid containing cellular material and alcohol are deposited within a pressure vessel or reactor. Elevated temperature and pressure applied within the vessel create a supercritical reaction condition. A co-solvent may also be deposited in the vessel if desired, as shown in FIG. 3. After a period of time at supercritical reaction conditions, a product is obtained. Excess alcohol remaining with the product can be evaporated and recycled as demonstrated in FIG. 3. After the alcohol is removed, fatty acid esters can be obtained as well as by-products and clean-up fuel, such as hydrocarbon components and other lipids. The fatty acid esters can be used to create a biofuel, such as biodiesel.

In another embodiment, a method is disclosed comprising a one step method as described herein, wherein the reactor contains a porous structure. The porous structure can create a greater surface area for reactions to occur within the reactor or vessel when operating at near critical or supercritical conditions. This can lessen stringent requirements on reactor or vessel design. The porous structure can be reticulated foam. The reticulated foam can be made of or coated with a nickel substance.

An additional benefit of the methods of the invention is the ready availability of an aqueous hydrolysate solution which can be of value for subsequent fermentation procedures or for can be used in animal feed or as a fertilizer. Fermentation of the aqueous solution, with subsequent extraction or distillation, can be readily conducted in such a manner as to yield additional valuable fuel products, such as ethanol, butanol, or acetone.

A vessel is provided herein in which any of the methods of the invention can be carried out. In many embodiments, the vessel is capable of withstanding elevated temperatures and pressures. In an embodiment, the vessel is capable of maintaining its integrity under supercritical reaction conditions within the vessel.

A vessel in which a method of the invention can occur can be made of materials such as stainless steel alloys, nickel alloys, titanium alloys, ceramics, glasses, or other materials known to be resistant to the effects of reactants at elevated temperatures and pressures.

The vessel can be of multiple geometries, such that the reaction can occur in the fashion known as “batch” processing, or as “continuous flow” processing. Thus the containment vessel may consist of forms such as tanks and spheres, cylinders, lengths of tubing, hollow fibers, and such. The design and fabrication of such reaction systems is well known to those involved with chemical processes.

Reactions in accordance with the invention may be conducted in continuous, batch, or semi-batch mode.

The invention also provides a system comprising a reactor containing a composition comprising cellular material, a means for elevating the temperature and pressure within the reactor, and an outlet for collecting fatty acid esters.

The reactor can be any reactor, vessel, or device capable of carrying out at least one portion of any method the invention described herein. The reactor can also be a vessel of the invention.

Means for elevating the temperature and pressure within the reactor can be separate from, coupled to, or part of the reactor. Many different methods of elevating temperature and pressure are known to those with skill in the art and can be used with a system of the invention. In many embodiments, means of elevating temperature and pressure are capable of creating near critical or supercritical reaction conditions within the reactor.

In an embodiment, the system of the invention comprises a vessel or reactor, a separator, and a product. The vessel is preferably a vessel of the invention. After a method of the invention is carried out in the vessel, the products (e.g. aqueous phase and oily phase) can be separated in a separator. The separated oily phase can then be deposited in another vessel. The second vessel can also be the same vessel that carried out the initial reaction. An outlet from the second vessel allows for collection of a product, such as fatty acid esters, fatty acids, and hydrocarbons.

An outlet for collecting fatty acid esters can be a valve, tube, or opening from which fatty acid esters can be obtained. The outlet may lead directly or indirectly to a purification method or system, such as those purification methods described herein, or those commonly known in the art. The outlet can provide a system of collecting fatty acid esters that can be directly converted to a fuel additive or fuel, such as biodiesel.

It should also be recognized that the methods, vessels, and systems of the invention can benefit those seeking to extract and concentrate hydrocarbons without further modification. Certain microorganisms are known to produce various hydrocarbons, which can also be readily obtained by methods disclosed herein.

### EXAMPLE 1

A living culture of Chlorella sp. microalgae was centrifuged at 1000 g. force for a period of 5 minutes. The resulting plug of cellular material was mixed with an equal volume of technical grade methanol, then transferred to a stainless steel cylindrical pressure vessel. The vessel was sealed with a threaded plug then placed in a 350° C. molten tin bath for 12 minutes. After cooling in a water bath for several minutes, the vessel was opened, and the brown solution within evaporated to dryness at room temperature. The residue was partitioned between hexane and water, and the hexane layer analyzed on a GCMS chromatograph. Analysis indicated the presence of predominantly C12-C20 fatty acid methyl esters, along with less than 10% of a mixture of fatty acids and monoglycerides. No unreacted triglycerides were detected.

### EXAMPLE 2

A 20% w/v slurry of mixed species microalgae and bacteria, originating from a sewage treatment lagoon, was pumped through a length of 6 mm inner diameter 316 stainless steel tubing, which was maintained at 340° C. by means of a surrounding cast aluminum cylinder, which was heated by electrical resistance cartridges. A system pressure of 20 MPa was maintained by means of an adjustable back pressure relief valve. The pumping rate was adjusted so as to allow for 16 minutes of residence time within the heated tubing. The output from the system consisted of a brown suspension, which upon 4 hours standing, separated into a less dense layer comprised nearly entirely of fatty acids along with minor amounts of hydrophobic degradation compounds, and an aqueous layer, which consisted mainly of amino acids, carbohydrates, minerals, and heterocyclic bases.

### EXAMPLE 3

A slurry of proprietary microalgae containing 0.58 grams (dry weight) of cells in 8 mls of water, was added, along with 3.5 mls technical grade hexane, to a stainless steel pressure vessel. The vessel was sealed and heated to 350° C. for 20 minutes, then cooled and opened. The hexane layer was combined with an equal volume of technical grade methanol, then sealed and reheated in the pressure vessel for an additional 20 minutes at 350° C. The resulting reaction mixture was dried at 80° C. until no further weight loss was noted. The residue weighed 0.24 grams and, upon GCMS chromatographic analysis, was shown to consist of a nearly pure mixture of C10-C22 fatty acid methyl esters. The yield of algae derived methyl esters was over 49%.

### EXAMPLE 4

7 mls of an aqueous slurry containing 0.39 grams of proprietary microalgae was combined with 3 mls of technical grade hexane and reacted in a stainless steel pressure vessel under 340° C. and 20 MPa conditions. Upon removal from the vessel, and separation and evaporation of the hexane layer, a residue consisting primarily of fatty acids was obtained in a quantity which equated to 39.9% of the original cellular mass.

An identical mass of the same dried algal material was extracted with a 3:2 v/v chloroform:methanol mixture according to the well known “Folsch” method, and a quantity of lipid like components amounting to 20.6% of the original cellular mass was obtained.

What is claimed is:

1. A method of producing fatty acid esters comprising: reacting a composition comprising cellular material in a reactor, wherein the temperature and pressure within the reactor are elevated such that the cellular material is destructible and components of the cellular material form an aqueous phase and an oily phase; and reacting the oily phase with an alcohol, thereby producing fatty acid esters.

2. The method of claim 1, wherein the cellular material is lipid-containing biomass from animals, plants, fungi, microalgae, macroalgae, bacteria, diatoms, or protozoa.

3. The method of claim 1, wherein the cellular material comprises at least 10% intact cells w/w based on the total weight of the cellular material.

4. The method of claim 1, wherein the composition contains at least 1%, 5%, 10%, 20%, or 50% water w/w based on the total weight of the composition.

5. The method of claim 1, wherein the composition contains between 10% and 90% cellular material w/w based on the total weight of the composition.

6. The method of claim 1, wherein elevating the temperature and pressure within the reactor creates a near critical or supercritical reaction condition.

7. The method of claim 6, wherein the temperature is elevated to between 180° C. and 450° C.

8. The method of claim 6, wherein the pressure is elevated to between 0.5 MPa and 40 MPa.

9. The method of claim 1, wherein the oily phase comprises at least one of fatty acids, monoglycerides, diglycerides, or triglycerides.

10. The method of claim 1, wherein the oily phase is reacted with the alcohol at a near critical or supercritical reaction condition.

11. The method of claim 1, wherein the alcohol has 1 to 20 carbon atoms.

12. The method of claim 11, wherein the alcohol is methanol or ethanol.

13. The method of claim 1, wherein the fatty acid ester is fatty acid methyl ester.

14. The method of claim 1, wherein the reaction is carried out in the presence of a co-solvent.

15. The method of claim 14, wherein the co-solvent is selected from the group consisting of carbon dioxide, nitrous oxide, sulfur dioxide, sulfur hexafluoride, alkanes and alkenes containing between 1 and 20 carbon atoms, alkyl halides, aromatic hydrocarbons, silicones, ethers, amines, alkyl oxides, and esters.

16. The method of claim 1, wherein the reaction is carried out in the presence of a catalyst.

17. The method of claim 16, wherein the catalyst is selected from the group consisting of: inorganic or organic acids or bases, metals or their oxides, silicates, carbonates or other salts of aluminum, magnesium, calcium, titanium, hafnium, nickel, silicon and zirconium.

18. The method of claim 1, further comprising: purifying the fatty acid esters produced.

19. A method of producing fatty acid esters comprising: reacting a composition comprising cellular material in a reactor, wherein the temperature and pressure within the reactor are elevated such that the cellular material is destructible and the components of cellular material form an aqueous phase and an oily phase; separating the aqueous phase from the oily phase; and reacting the oily phase with an alcohol, thereby producing a fatty acid ester.

20. The method of claim 19, wherein the cellular material is lipid-containing biomass from animals, plants, fungi, microalgae, macroalgae, bacteria, diatoms, or protozoa.

21. The method of claim 19, wherein the cellular material comprises at least 10% intact cells w/w based on the total weight of the cellular material.

22. The method of claim 19, wherein the composition contains at least 1%, 5%, 10%, 20%, or 50% water w/w based on the total weight of the composition.

23. The method of claim 19, wherein the composition contains between 10% and 90% cellular material w/w based on the total weight of the composition.

24. The method of claim 19, wherein elevating the temperature and pressure within the reactor creates a near critical or supercritical reaction condition.

25. The method of claim 24, wherein the temperature is elevated to between 180° C. and 450° C.

26. The method of claim 24, wherein the pressure is elevated to between 0.5 MPa and 40 MPa.

27. The method of claim 19, wherein the oily phase comprises at least one of fatty acids, monoglycerides, diglycerides, or triglycerides.

28. The method of claim 19, wherein the oily phase is reacted with the alcohol at a near critical or supercritical reaction condition.

29. The method of claim 19, wherein the alcohol has 1 to 20 carbon atoms.

30. The method of claim 29, wherein the alcohol is methanol or ethanol.

31. The method of claim 19, wherein the fatty acid ester is fatty acid methyl ester.

32. The method of claim 19, wherein the reaction is carried out in the presence of a co-solvent.

33. The method of claim 32, wherein the solvent is selected from the group consisting of: carbon dioxide, nitrous oxide, sulfur dioxide, sulfur hexafluoride, alkanes and alkenes containing between 1 and 20 carbon atoms, alkyl halides, aromatic hydrocarbons, silicones, ethers, amines, alkyl oxides, and esters.

34. The method of claim 19, wherein the reaction is carried out in the presence of a catalyst.

35. The method of claim 34, wherein the catalyst is selected from the group consisting of inorganic or organic acids or bases, metals or their oxides, silicates, carbonates or other salts of aluminum, magnesium, calcium, titanium, hafnium, nickel, silicon and zirconium.

36. The method of claim 19, further comprising: purifying the fatty acid esters produced.

37. The method of claim 19, wherein the separating is accomplished by process selected from the group consisting of settling, gravity separation, centrifugal separation, filtration, and extraction.

38. A method of producing fatty acid esters comprising: reacting a composition comprising cellular material in a reactor with an aqueous solution, wherein the temperature and pressure within the reactor are elevated such that the cellular material is destructible and components of the cellular material form an aqueous phase and an oily phase; and reacting the oily phase with an alcohol, thereby producing a fatty acid ester.

39. The method of claim 38, wherein the cellular material is lipid-containing biomass from animals, plants, fungi, microalgae, macroalgae, bacteria, diatoms, or protozoa.

40. The method of claim 38, wherein the cellular material comprises at least 10% intact cells w/w based on the total weight of the cellular material.

41. The method of claim 38, wherein the composition contains at least 1%, 5%, 10%, 20%, or 50% water w/w based on the total weight of the composition.

42. The method of claim 38, wherein the composition contains between 10% and 90% cellular material w/w based on the total weight of the composition.

43. The method of claim 38, wherein elevating the temperature and pressure within the reactor creates a near critical or supercritical reaction condition.

44. The method of claim 43, wherein the temperature is elevated to between 180° C. and 450° C.

45. The method of claim 43, wherein the pressure is elevated to between 0.5 MPa and 40 MPa.

46. The method of claim 38, wherein the oily phase comprises at least one of fatty acids, monoglycerides, diglycerides, or triglycerides.

47. The method of claim 38, wherein the oily phase is reacted with the alcohol at a near critical or supercritical reaction condition.

48. The method of claim 38, wherein the alcohol has 1 to 20 carbon atoms.

49. The method of claim 48, wherein the alcohol is methanol or ethanol.

50. The method of claim 38, wherein the fatty acid ester is fatty acid methyl ester.

51. The method of claim 38, wherein the reaction is carried out in the presence of a co-solvent.

52. The method of claim 51, wherein the co-solvent is selected from the group consisting of: carbon dioxide, nitrous oxide, sulfur dioxide, sulfur hexafluoride, alkanes and alkenes containing between 1 and 20 carbon atoms, alkyl halides, aromatic hydrocarbons, silicones, ethers, amines, alkyl oxides, and esters.

53. The method of claim 38, wherein the reaction is carried out in the presence of a catalyst.

54. The method of claim 53, wherein the catalyst is selected from the group consisting of: inorganic or organic acids or bases, metals or their oxides, silicates, carbonates or other salts of elements such as the alkali elements including aluminum, magnesium, calcium, titanium, hafnium, nickel, silicon and zirconium.

55. The method of claim 38, further comprising: purifying the fatty acid esters produced.

56. The method of claim 38, wherein the aqueous solution is water.

57. The method of claim 38, wherein the aqueous solution is between 5% and 90% of the total reaction composition.

58. A method of producing fatty acid esters comprising: reacting a composition comprising cellular material in the presence of alcohol in a reactor, wherein the temperature and pressure within the reactor are elevated such that the cellular material is destructible and wherein the alcohol reacts with the oily components of the cellular material, thereby producing fatty acid esters.

59. The method of claim 58, wherein the cellular material is lipid-containing biomass from animals, plants, fungi, microalgae, macroalgae, bacteria, diatoms, or protozoa.

60. The method of claim 58, wherein the cellular material comprises at least 10% intact cells w/w based on the total weight of the cellular material.

61. The method of claim 58, wherein the composition contains at least 1%, 5%, 10%, 20%, or 50% water w/w based on the total weight of the composition.

62. The method of claim 58, wherein the composition contains between 10% and 90% cellular material w/w based on the total weight of the composition.

63. The method of claim 58, wherein elevating the temperature and pressure within the reactor creates a near critical or supercritical reaction condition.

64. The method of claim 63, wherein the temperature is elevated to between 180° C. and 450° C.

65. The method of claim 63, wherein the pressure is elevated to between 0.5 MPa and 40 MPa.

66. The method of claim 58, wherein the oily phase comprises at least one of fatty acids, monoglycerides, diglycerides, or triglycerides.

67. The method of claim 58, wherein the oily phase is reacted with the alcohol at a near critical or supercritical reaction condition.

68. The method of claim 58, wherein the alcohol has 1 to 20 carbon atoms.

69. The method of claim 68, wherein the alcohol is methanol or ethanol.

70. The method of claim 58, wherein the fatty acid ester is fatty acid methyl ester.

71. The method of claim 58, wherein the reaction is carried out in the presence of a co-solvent.

72. The method of claim 71, wherein the co-solvent is selected from the group consisting of: carbon dioxide, nitrous oxide, sulfur dioxide, sulfur hexafluoride, alkanes and alkenes containing between 1 and 20 carbon atoms, alkyl halides, aromatic hydrocarbons, silicones, ethers, amines, alkyl oxides, and esters.

73. The method of claim 58, wherein the reaction is carried out in the presence of a catalyst.

74. The method of claim 73, wherein the catalyst is selected from the group consisting of: inorganic or organic acids or bases, metals or their oxides, silicates, carbonates or other salts of elements aluminum, magnesium, calcium, titanium, hafnium, nickel, silicon and zirconium.

75. The method of claim 58, further comprising purifying the fatty acid esters produced.

76. A method of producing fatty acid esters comprising: reacting a composition comprising cellular material in the presence of alcohol in a reactor, wherein the reactor comprises a container containing a porous structure and wherein the temperature and pressure within the reactor are elevated such that the cellular material is destructible and wherein the alcohol reacts with the oily components of the cellular material, thereby producing fatty acid esters.

77. The method of claim 76, wherein the cellular material is lipid-containing biomass from animals, plants, fungi, microalgae, macroalgae, bacteria, diatoms, or protozoa.

78. The method of claim 76, wherein the cellular material comprises at least 10% intact cells w/w based on the total weight of the cellular material.

79. The method of claim 76, wherein the composition contains at least 1%, 5%, 10%, 20%, or 50% water w/w based on the total weight of the composition.

80. The method of claim 76, wherein the composition contains between 10% and 90% cellular material w/w based on the total weight of the composition.

81. The method of claim 76, wherein elevating the temperature and pressure within the reactor creates a near critical or supercritical reaction condition.

82. The method of claim 81, wherein the temperature is elevated to between 180° C. and 450° C.

83. The method of claim 81, wherein the pressure is elevated to between 0.5 MPa and 40 MPa.

84. The method of claim 76, wherein the oily phase comprises at least one of fatty acids, monoglycerides, diglycerides, or triglycerides.

85. The method of claim 76, wherein the oily phase is reacted with the alcohol at a near critical or supercritical reaction condition.

86. The method of claim 76, wherein the alcohol has 1 to 20 carbon atoms.

87. The method of claim 86, wherein the alcohol is methanol or ethanol.

88. The method of claim 76, wherein the fatty acid ester is fatty acid methyl ester.

89. The method of claim 76, wherein the reaction is carried out in the presence of a co-solvent.

90. The method of claim 89, wherein the co-solvent is selected from the group consisting of: carbon dioxide, nitrous oxide, sulfur dioxide, sulfur hexafluoride, alkanes and alkenes containing between 1 and 20 carbon atoms, alkyl halides, aromatic hydrocarbons, silicones, ethers, amines, alkyl oxides, and esters.

91. The method of claim 76, wherein the reaction is carried out in the presence of a catalyst.

92. The method of claim 91, wherein the catalyst is selected from the group consisting of: inorganic or organic acids or bases, metals or their oxides, silicates, carbonates or other salts of elements such as the alkali elements including aluminum, magnesium, calcium, titanium, hafnium, nickel, silicon and zirconium.

93. The method of claim 76, further comprising: purifying the fatty acid esters produced.

94. The method of claim 76, wherein the porous structure is reticulated foam.

95. A vessel for carrying out the method of claim 1, 19, 38, 58, or 76.

96. The vessel of claim 95, wherein the vessel is a batch processing vessel.

97. The vessel of claim 95, wherein the vessel is a continuous flow processing vessel.

98. A system comprising:
(a) a reactor containing a composition comprising cellular materials;
(b) a means for elevating the temperature and pressure within the reactor; and
(c) an outlet for collecting fatty acid esters.
